(19) United States
(12) Patent Application Publication
(10) Pub. No.: US 2019/0183856 A1
(43) Pub. Date: Jun. 20, 2019

(54) METHODS FOR TREATING CANCER

(71) Applicant: Macau University of Science and Technology, Macau (CN)

(72) Inventors: Wenzhe MA, Macau (CN); Ying XIE, Macau (CN); Xiaolin LIAO, Macau (CN); Jiajun HUANG, Macau (CN); Wanjun LIN, Macau (CN); Ze LONG, Macau (CN)

(21) Appl. No.: 15/846,129

(22) Filed: Dec. 18, 2017

(51) Int. Cl. A61K 31/381; A61P 35/00; G01N 33/574

(52) U.S. Cl. CPC A61K 31/381; G01N 2333/4748; G01N 33/57419; A61P 35/00

(57) ABSTRACT

One embodiment relates to a method of treating cancer by administering a compound of Formula I to a patient. Another embodiment relates to a method of treating a patient having cancer associated p53 mutation that includes administering to a patient the compound represented by the Formula I:

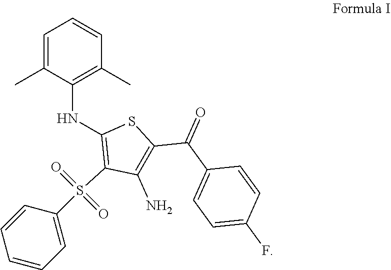 [SMILES: Cc1cccc(C)c1Nc1sc(C(=O)c2ccc(F)cc2)c(N)c1S(=O)(=O)c1ccccc1]

### FIELD OF THE INVENTION

The present invention relates to a method of treating cancer. In particular, the present invention relates to a method of treating colon cancer.

### BACKGROUND OF INVENTION

Cancer is a disease involving abnormal cell growth with the potential to invade or spread to other areas of the body. Colon cancer is one of the most common human tumors in developed and developing countries. It is estimated that 1.361 million people were diagnosed with and 0.694 million people died of colon cancer worldwide in 2012.

In view of the demand for effectively treating cancer, particularly colon cancer, improvements in method are desired.

### SUMMARY OF INVENTION

One example embodiment is a method of treating a cancer in a patient in need thereof. The method includes administering a therapeutically effective amount of a compound to the patient to treat the cancer, the compound is represented by Formula I:

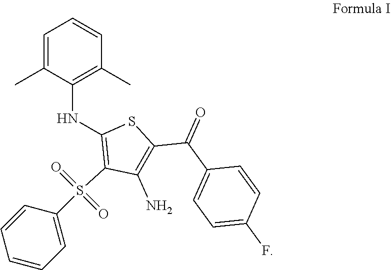 [SMILES: Cc1cccc(C)c1Nc1sc(C(=O)c2ccc(F)cc2)c(N)c1S(=O)(=O)c1ccccc1]

Another example embodiment is a method of treating cancer in a patient in need thereof. The method includes diagnosing the patient having cancer associated p53 mutation; classifying the patient as Group 1 if the diagnosis of p53 mutation is positive, and classifying the patient as Group 2 if the diagnosis of p53 mutation is negative; and administering a therapeutically effective amount of a compound to the patient in Group 2 to treat the cancer, and the compound is represented by Formula I.

Other example embodiments are discussed herein.

### BRIEF DESCRIPTION OF FIGURES

The patent or application file contains at least one drawing executed in color. Copies of this patent or patent application publication with color drawing(s) will be provided by the Office upon request and payment of the necessary fee.

FIG. 1a shows a dose effect of 48 hours APTM treatment on the proliferation of HCT116 cell in accordance with an example embodiment. The cell number at each dose point is presented as a percentage of control. Average values are from three independent experiments performed in triplicate (n=3).

FIG. 1b shows a time course of APTM treatment (10 μM) on the proliferation of HCT116 cell in accordance with an example embodiment. The cell number at each time point is presented as a percentage of control. Average values are from three independent experiments conducted separately.

FIG. 1c shows an effect of APTM on colony formation in HCT116 cells in accordance with an example embodiment. Representative colony formation assay plates of HCT116 cells treated with indicated concentrations of APTM are shown in the left. The quantification of colony number (n=3) are in the right. Data are shown as mean±SD.

FIG. 2 shows cell cycle distributions of HCT116 cells after treatment with APTM at indicated concentrations for 48 hours analyzed by flow cytometry in accordance with an example embodiment. It indicated APTM does not induce cell cycle arrest in HCT116 cells as shown in the right. Average values are from three independent experiments (n=3). Data are shown as mean±SD. It also indicated an increase of apoptotic cells as shown in the left in accordance with an example embodiment.

FIG. 3a shows that visualization of apoptotic morphological changes by a fluorescent microscope with Hoechst 33258 staining after HCT116 cells are treated with APTM at indicated concentrations for 48 hours in accordance with an example embodiment. Representative pictures are shown (400×).

FIG. 3b shows that cells were stained with annexin V-FITC/PI and apoptosis were tested by flow cytometry in accordance with an example embodiment. Representative contour diagrams are shown.

FIG. 3c shows quantified fractions of apoptotic cells in accordance with an example embodiment. Average values are from three independent experiments (n=3).

FIG. 3d shows a western blotting analysis of apoptosis marker p53, Bax and cleaved nuclear poly(ADP-ribose) polymerase (cPARP) in accordance with an example embodiment. Data are shown as mean±SD.

FIG. 4a shows that visualization of apoptotic morphological changes by a fluorescent microscope with Hoechst 33258 staining after HCT116 p53−/− cells were treated with APTM at indicated concentrations for 48 hours in accordance with an example embodiment. Representative pictures are shown (400×).

FIG. 4b shows that cells were stained with Annexin V-FITC/PI and apoptosis were tested by flow cytometry in accordance with an example embodiment. Representative contour diagrams are shown.

FIG. 4c shows a western blotting analysis of apoptosis marker p53, Bax and cleaved nuclear poly(ADP-ribose) polymerase (cPARP) in accordance with an example embodiment.

FIG. 4d shows a cell growth curve in accordance with an example embodiment. The cell number at each dose is presented as a percentage of control. Average values are from three independent experiments conducted separately. Data are shown as mean±SD.

FIG. 5 shows a method to treat cancer in a patient in need of such treatment in accordance with an example embodiment.

FIG. 6 shows a method to treat cancer in a patient in need of such treatment in accordance with an example embodiment.

### DETAILED DESCRIPTION OF THE PREFERRED EMBODIMENTS

Example embodiments relate to methods of treating a cancer in a patient in need thereof. The methods comprise administering a therapeutically effective amount of a compound of [3-Amino-5-[(2,6-dimethylphenyl)amino]-4-(phenylsulfonyl)-2-thienyl](4-fluorophenyl)methanone (APTM). APTM is represented by below Formula I.

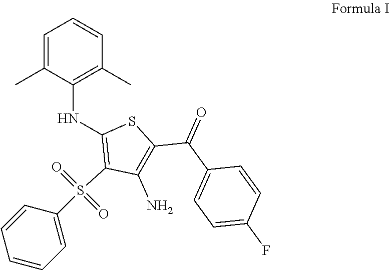 [SMILES: Cc1cccc(C)c1Nc1sc(C(=O)c2ccc(F)cc2)c(N)c1S(=O)(=O)c1ccccc1]

Example embodiments relate to methods of treating cancer in a patient in need thereof. The methods comprise diagnosing if the patient having cancer associated p53 mutation; classifying the patient as Group 1 if the diagnosis of p53 mutation is positive, and classifying the patient as Group 2 if the diagnosis of p53 mutation is negative; administering a therapeutically effective amount of a compound to the patient in Group 2 to treat the cancer, and the compound is represented by Formula I.

Example embodiments relate to a pharmaceutical composition comprising a compound and a pharmaceutically acceptable excipient. The compound is represented by Formula I.

In one example embodiment, the cancer is colon cancer. In another example embodiment, the patient does not has p53 mutation. In a further embodiment for example, the p53 mutation is deletion of p53.

[3-Amino-5-[(2,6-dimethylphenyl)amino]-4-(phenylsulfonyl)-2-thienyl](4-fluorophenyl)methanone (APTM) is a synthesized thiophene heterocyclic compound with a high activity and novel structure.

### Example 1

### Material and Methods

### 1. Chemicals and Drugs

APTM was dissolved in dimethylsulfoxide (DMSO) and stored at −40° C. until use. Sulforhodamine B (SRB), trichloroacetic acid (TCA), crystal violet and Hoechst 33258 were obtained from Sigma Aldrich. McCoy's 5A (Modified) medium, fetal bovine serum (FBS), TrypLE™ Express enzyme and penicillin-streptomycin (10,000 U/mL) were purchased from GIBCO. Annexin V: FITC Apoptosis Detection Kit I was purchased from BD Biosciences and cell cycle detection kit was purchased from Nanjing Key GEN BioTECH. The primary antibodies for cleaved nuclear poly (ADP-ribose) polymerase (cPARP), p53 and Bax were purchased from Cell Signaling. β-actin was purchased from Sigma. Horseradish peroxidase-conjugated secondary antibodies were purchased from Jackson ImmunoResearch Inc.

### 2. Cell Lines and Culture

Human colon cancer cell line HCT116 (p53+/+) was purchased from ATCC and the p53−/− HCT116 cell line was provided by Bert Vogelstein, Johns Hopkins University. Cells were cultured in McCoy's 5A medium supplemented with 10% FBS, 100 units/ml penicillin G, and 100 μg/ml streptomycin in humidified atmosphere with 5% CO₂. Cells were passaged twice weekly to maintain logarithmic growth.

### 3. In Vitro Cell Proliferation Assay (SRB Assay)

The anti-proliferative effects of APTM on cancer cell lines were assessed by sulforhodamine B (SRB) colorimetric assay. Cells were seeded in 96-well plates at densities of 5,000 cells per well and cultured overnight. Then cells were susceptible to APTM at indicated concentrations and cultured for indicated times. After incubation, attached cells were fixed with 50 μL cold 50% (w/v) trichloroacetic acid (TCA) for 1 hour at 4° C., washed 5 times with slow-running tap water, and stained with 100 μL 0.4% (w/v) SRB. Optical density at 515 nm (OD₅₁₅) was measured using a microplate reader (Molecular Devices) after mixing the protein-bound dye with 200 μL 10 mM Tris base solution (pH 10.5). The relative cell growth rate was determined with the following equation: Relative Growth (%)=OD (treated)/OD (control). The IC₅₀ value was defined as the concentration required for a 50% reduction in cell growth.

### 4. Colony Formation Assay

HCT116 cells were plated at 3,000 cells/well in six-well plates and cultured with indicated concentrations of APTM for 8 days. Cells were stained with 0.2% (w/v) crystal violet in buffered formalin for 20 minutes, and colonies were then photographed and quantified as previously described.

### 5. Analysis of Cell Cycle by Flow Cytometry

Cell cycle distribution was determined by staining DNA with propidium iodide (PI). Briefly, 1.0×10⁶ cells were incubated with or without APTM for 48 hours. Cells were washed with cold PBS and fixed in 70% ethanol at −20° C. for 2 hours. After washing with PBS, cells were stained with cold PI solution (20 μg/ml PI and 200 μg/mI RNase in PBS) for 30 minutes at room temperature in the dark. The percentage of cells in different phases of the cell cycle was determined by flow cytometer (BD Bioscience) and analyzed using FlowJo software.

### 6. Analysis of Apoptosis by Flow Cytometry

Annexin V-FITC/PI double staining method was employed for the apoptosis assay in HCT116 p53+/+ and HCT116 p53−/− cells (1×10⁶/well, 6-well plate). After 48 hours of treatment with APTM, cells were harvested in 15 ml centrifuge tubes by gentle scraping followed by centrifugation at 300×g for 5 minutes at room temperature. Cell suspension was washed two times with cold PBS by centrifugation at 300×g for 5 minutes at room temperature. Then cells were harvested, washed twice with cold PBS, and resuspended in 1×binding buffer (100 μL). Cells were transferred into a 1.5 ml micro-centrifuge tubes and stained with propidium iodide (5 μL) and FITC annexin V (5 μL). Cells were briefly vortexed after incubation for 15 minutes in the dark at room temperature. Then cells were filtered and analyzed by flow cytometry. Total apoptotic cells (FITC to Annexin V positive) were counted.

### 7. Assessment of Cell Morphological Changes

Cells were plated in 6-well plates (200,000 cells/well) and treated with indicated concentrations of APTM. After incubation for 48 hours, cells were collected, washed with PBS and stained with Hoechst 33258 (11.1 g/ml) in buffered formalin solution containing 5.6% NP-40. Apoptotic and living cells were viewed through DAPI filter of fluorescence inverted microscope (Leica DM2500 Fluorescence Microscope) at 400× magnifications.

### 8. Western Blotting

HCT116 cells were treated with APTM at indicated concentrations for 48 hours, and harvested via trypsinization. Protein samples were prepared by scratching cells in RIPA buffer containing protease inhibitor cocktail (Roche) and diluted in SDS-PAGE protein sample buffer. Samples were heated for 5 minutes at 100° C. Protein concentrations were measured using Direct Detect® Infrared Spectrometer (Millipore, USA) according to the manufacturer's instructions. Equal amount of proteins were loaded on 4-20% SDS-polyacrylamide (PAGE) gel. After electrophoresis, gels were transferred to a PVDF membrane (Millipore) and incubated with primary antibodies overnight at 4° C. The membranes were then washed with TBST and incubated with horseradish peroxidase-conjugated anti-rabbit or anti-mouse antibodies (1:10000, Santa Cruz, Calif., USA) for 45 min at room temperature. Proteins were visualized with SuperSignal West Dura Extended Duration Substrate or SuperSignal West Pico Chemiluminescent Substrate (Thermo Scientific) using the Amersham Imager 600 western blotting system.

### Example 2

The in vitro effect of APTM on the proliferation of human colon cancer HCT116 cells and the growth inhibition effect thereof are studied. By employing the p53 deficient HCT116 cells, it was found that induction of apoptosis by APTM is p53 dependent.

### 1. APTM Inhibits Proliferation of HCT116 Cells

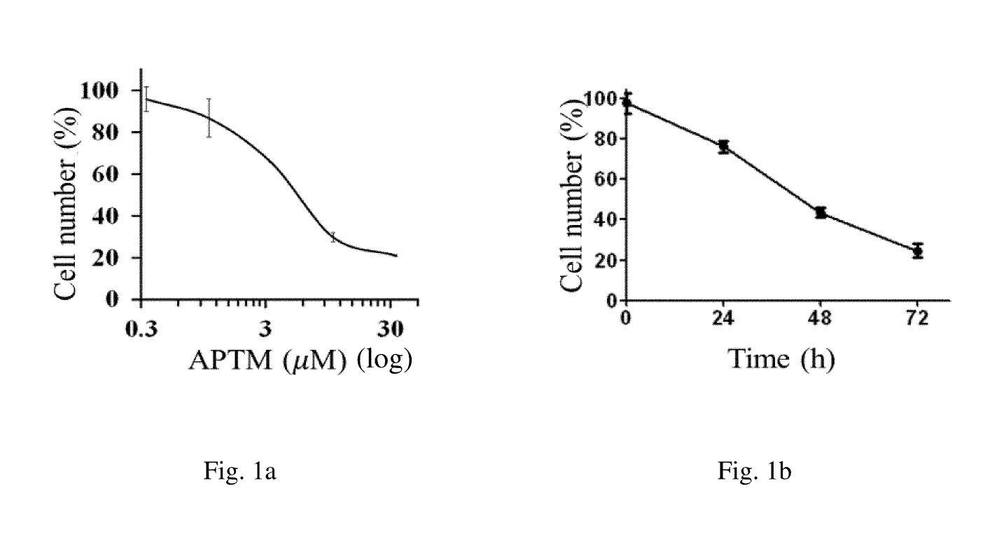

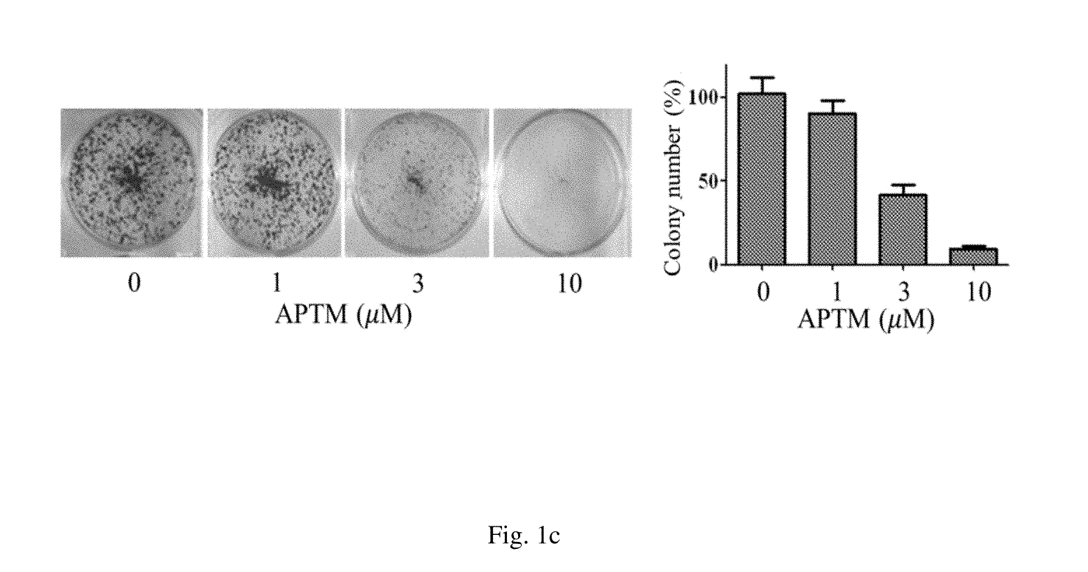

The proliferative effect of APTM on human colon cancer cell line HCT116 was examined using SRB assay. HCT116 cells were treated with a series of APTM concentrations of 0.33 μM, 3.33 μM and 33.3 μM for 48 hours. As shown in FIG. 1a, APTM reduced cell viability of HCT116 cells in a concentration-dependent manner, with IC₅₀ value of 6.57 μM. HCT116 cells were then treated with 10 μM of APTM and cytotoxicity was assessed at 24 hours, 48 hours and 72 hours after the treatment. Consistent with the dose effect of APTM, the growth of HCT116 cells was also inhibited in a time-dependent manner as shown in FIG. 1b. After treatment for 24 hours, the relative growth rate of HCT116 cells was 76.36% and declined to 24.57% after 72 hours. It indicated that APTM inhibited the growth of human colon cancer cells HCT116 both concentration- and time-dependently. This was further proved by long time colony formation assay as shown in FIG. 1c. Colony formation ability of HCT116 cells was significantly decreased after treatment with APTM at 3.0 μM and almost vanished at 10.0 μM. These results suggest that APTM is potent in inhibiting the proliferation of HCT116 cells in vitro.

2. APTM does not Inhibit Cell Cycle Progression in HCT116 Cells

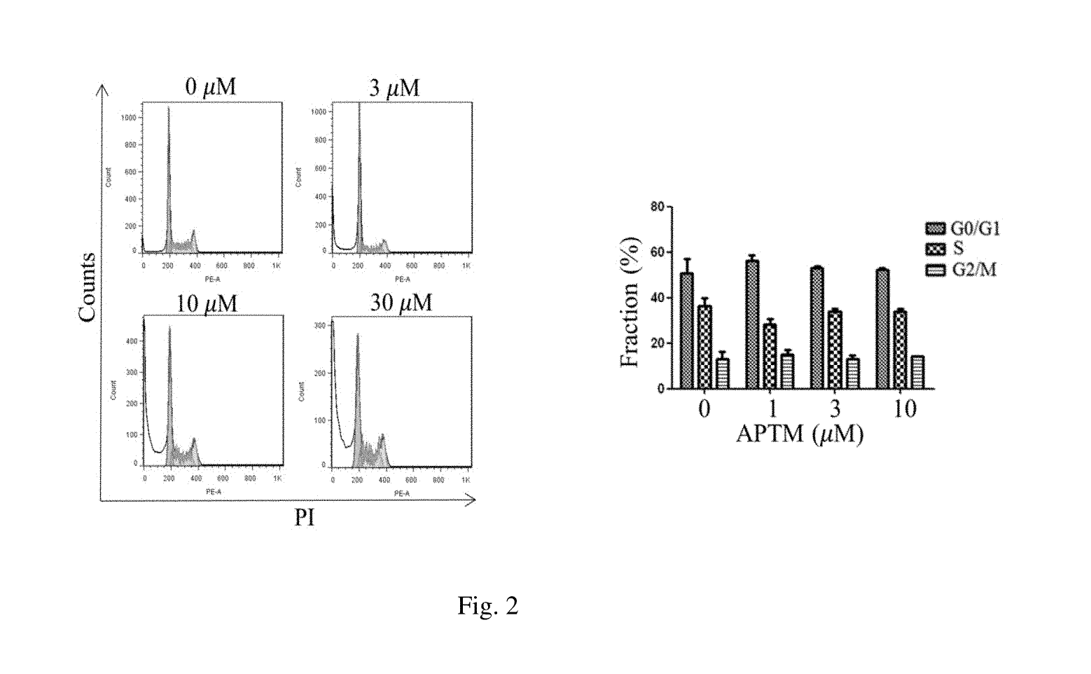

Cell cycle arrest is a key intracellular event contributing to reduced cell proliferation. It was checked whether APTM could alter cell cycle progression on colon cancer cells. Cell cycle distribution of HCT116 cells treated with APTM was analyzed by flow cytometry. As shown in FIG. 2, the proportions of cells in G0/G1, S and G2/M phases were almost unchanged after APTM treatment at different concentrations for 48 hours. It suggests that the reduced cell proliferation with APTM treatment is not due to the capture of cell cycle. On the other hand, the sub-G0/G1 fraction of cells was increased after APTM treatment in a concentration-dependent manner as shown in FIG. 2. As cells in sub-G0/G1 fraction represent apoptotic cells shown in FIG. 2, it reasonably suggested that APTM inhibits cell proliferation through induction of apoptosis.

### 3. APTM Induces Apoptosis in HCT116 Cells

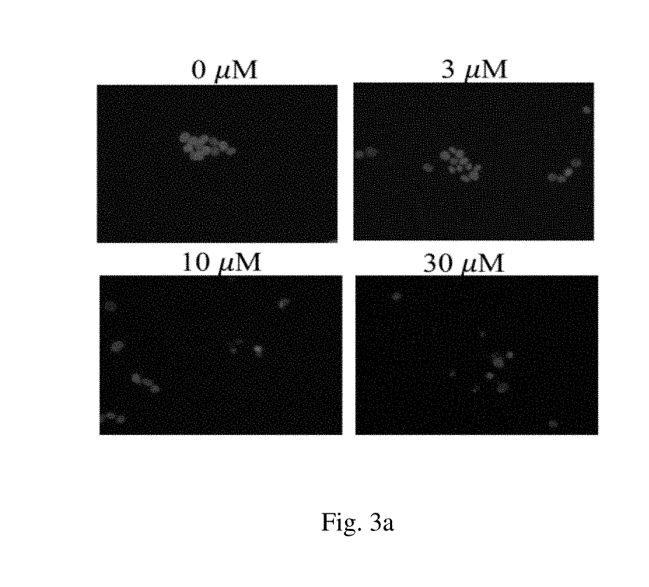

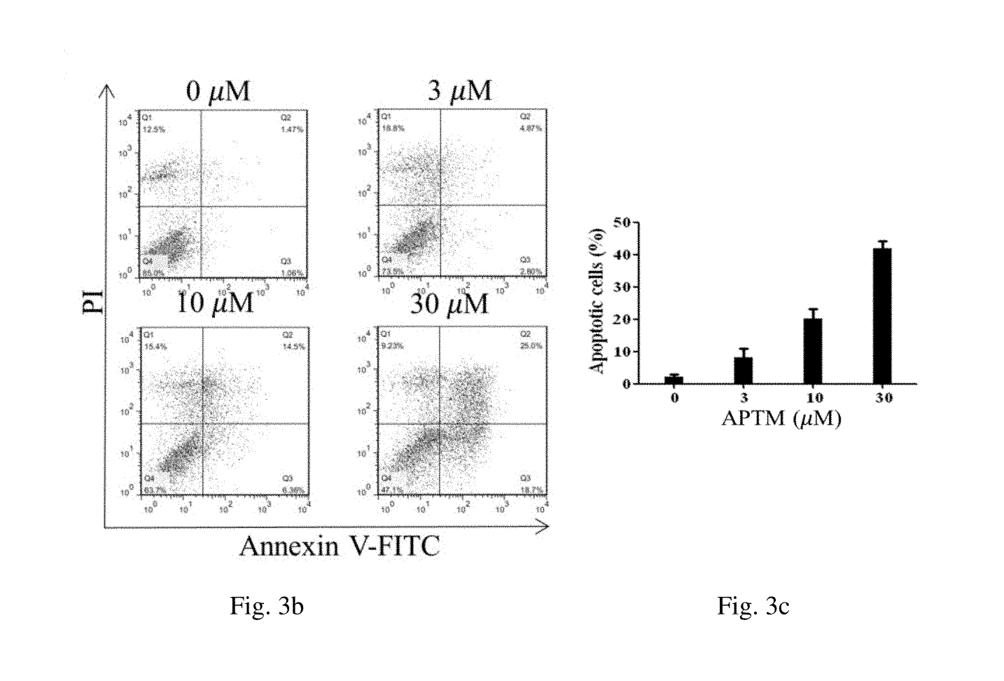

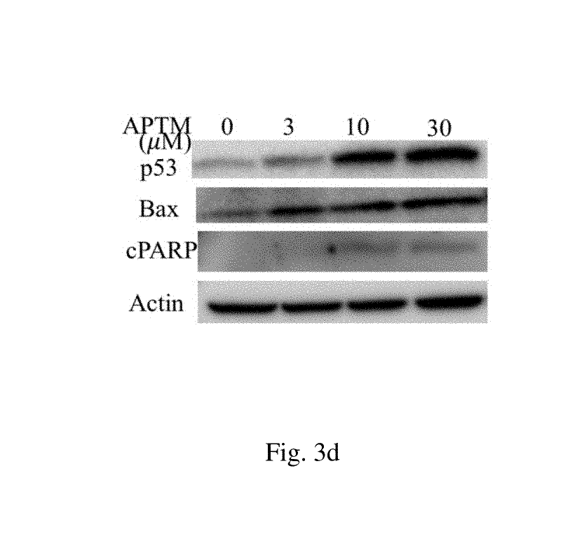

To further elucidate whether the growth inhibitory activity of APTM resulted from induction of apoptosis, HCT116 cells treated with 0, 3, 10 and 30 μM of APTM for 48 hours were stained with Hoechst and visualized under a fluorescent microscope. As shown in FIG. 3a, cells treated with APTM exhibits typical morphological characteristics of apoptosis, like nuclear fragmentation and chromatin condensation. This was further confirmed by flow cytometry analysis of cells stained with annexin V-FITC/PI as shown in FIG. 3b. APTM induced apoptosis of HCT116 cells in a concentration-dependent manner. The percentages of apoptotic cells (annexin V positive) with APTM treatment for 48 hours were 7.67% at 3 μM, 20.86% at 10 μM, and 43.7% at 30 μM, comparing with 2.53% of the control as shown in FIG. 3c.

The results were further demonstrated by a concentration-dependently induction of cleaved nuclear poly(ADP-ribose) polymerase (cPARP) with APTM treatment as shown in FIG. 3d. Cleavage of PARP by caspases is a key process during apoptosis and thus cPARP is a well-known apoptosis marker. It was found that Bax, a pro-apoptotic BCL2 family member, was also induced by APTM treatment as shown in FIG. 3c. Upon apoptotic stimuli, Bax undergoes conformational changes and oligomerizes at the mitochondrial outer membrane to promote its permeabilization and the releasing of cytochrome C and consequently activate caspases cascade. Since Bax is a known target of p53, a tumor suppressor mediating a variety of stress responses, it was reasonably suggested that APTM induces apoptosis through upregulation of p53 protein. Indeed, p53 protein was substantially induced by APTM treatment as shown in FIG. 3d. Therefore, it is indicated that APTM induces apoptosis in human colon cancer HCT116 cells through activation of the p53/Bax/cPARP signaling axis.

### 4. Depletion of p53 Attenuates APTM Effect on Apoptosis and Cell Proliferation

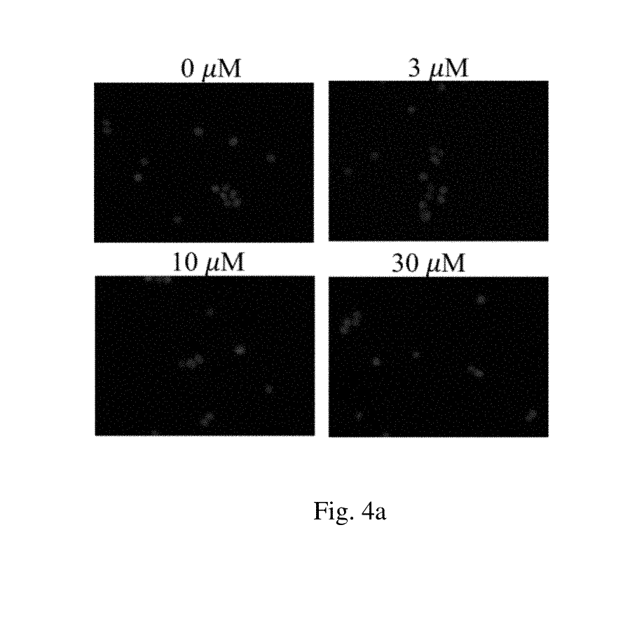

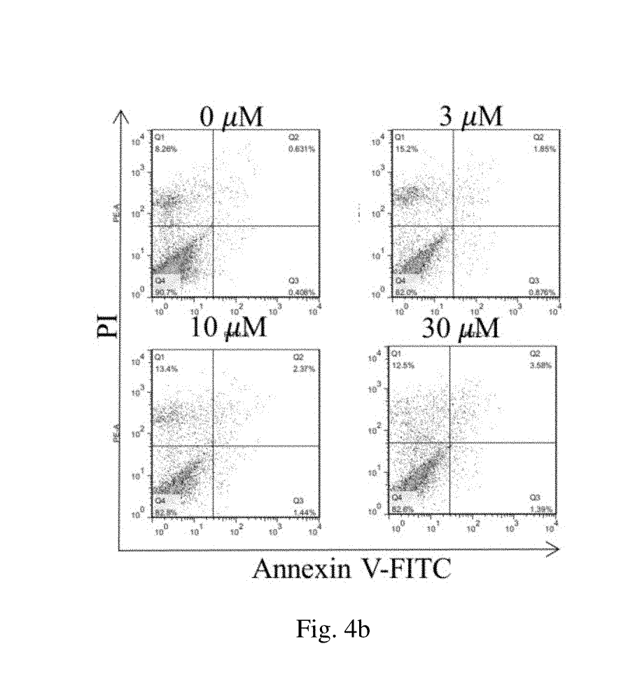

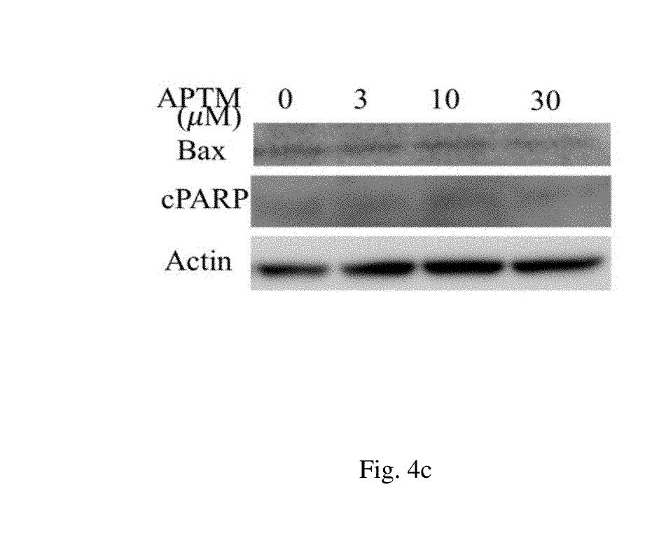

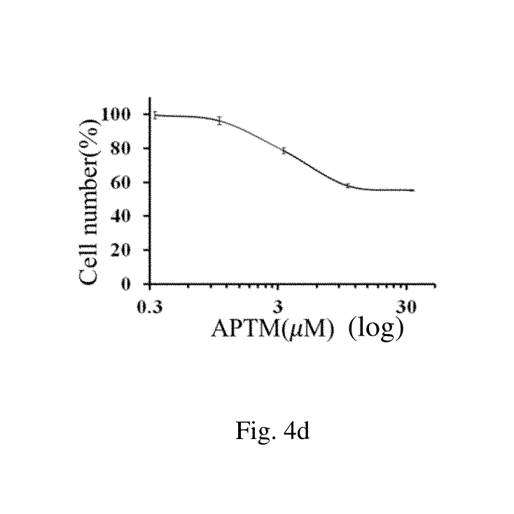

To test whether the effect of APTM on apoptosis induction and cell proliferation inhibition is dependent on p53, an isogenic HCT116 cell line lacking p53 (HCT116 p53−/−) is employed for further studies. Comparing with wild type HCT116 cells (HCT116 p53+/+), which showed apoptotic morphological changes with APTM treatment (as shown in FIG. 3a), HCT116 p53−/− cells reserved normal nuclear staining even at a highest concentration of 30 μM APTM [inventor, we understand the highest concentration used in this study is 33.3 μM, please confirm] as shown in FIG. 4a. Consistent with these results, HCT116 p53−/− cells were resistant to APTM induced apoptosis as analyzed by flow cytometry with annexin V-FITC/PI staining as shown in FIG. 4b. It indicated that p53 protein is essential for APTM induced apoptosis in human colon cancer HCT116 cells. This was subsequently verified by western blotting analysis in FIG. 4c, as loss of p53 blunted Bax and cPARP expression at different APTM concentrations. An anti-proliferative activity of APTM on HCT116 p53−/− cells was also tested using SRB assay. As shown in FIG. 4d, the IC₅₀ value of APTM on HCT116 p53−/− cells was greater than 33.3 μM, which is the highest concentration tested in this study. Comparing with the IC₅₀ value of 6.57 μM on HCT116 p53+/+ cells (as shown in FIG. 1b), there was a more than 5-fold resistance to APTM of the p53 deficient cells. These results indicated that the apoptosis induction and growth inhibition activity of APTM are p53-dependent.

Under physiologic conditions, p53 is negatively regulated by E3 ubiquitin ligase MDM2 and maintained at low levels. While in response to a wide range of stress stimuli, such as DNA damage, oxidative stress, nutrient deprivation, oncogene expression and hypoxia, the interaction between p53 and MDM2 is perturbed and p53 protein is stabilized. Once stabilized, p53 stand as the “guardian of the genome” through induction of apoptosis, cell cycle arrest, DNA repair and senescence.

The anti-proliferative effect of APTM on colon cancer HCT116 cells with an IC₅₀ value of 6.57 μM is studied. The growth inhibitory effect of APTM on colon cancer cells is confirmed by time course experiment and colony formation assay. It further shows that APTM induces p53 protein concentration-dependently in HCT116 p53+/+ cells.

One of the most remarkable functions of p53 is the induction of apoptosis. This occurs through either the extrinsic death receptor pathway, transcription dependent intrinsic mitochondrial pathway, or transcription independent cytosolic pathway. The pro-apoptotic protein Bax was induced with p53 protein following APTM treatment (as shown in FIG. 3d), and the induction of Bax was diminished in HCT116 p53−/− cells (as shown in FIG. 4c). So, it reasonably suggested that APTM promotes apoptosis through the transcription dependent mitochondrial pathway of p53. That is to say, p53 protein stabilized upon APTM treatment, increases Bax expression transcriptionally and promotes its oligomerization and mitochondrial translocation. Bax oligomers insert into mitochondrial outer membrane, and promote the releasing of cytochrome C leading caspases activation and apoptosis. On the other hand, p53 induction by APTM did not markedly change cell cycle distribution of HCT116 p53+/+ cells, as shown in FIG. 2. This selective induction of apoptosis instead of cell cycle arrest may be because of high levels of p53 following APTM treatment, since high level of p53 promotes apoptosis while low level thereof leads to cell cycle arrest. It is also possible that APTM affects cofactors or other proteins that can cooperate with p53 and alter its functions.

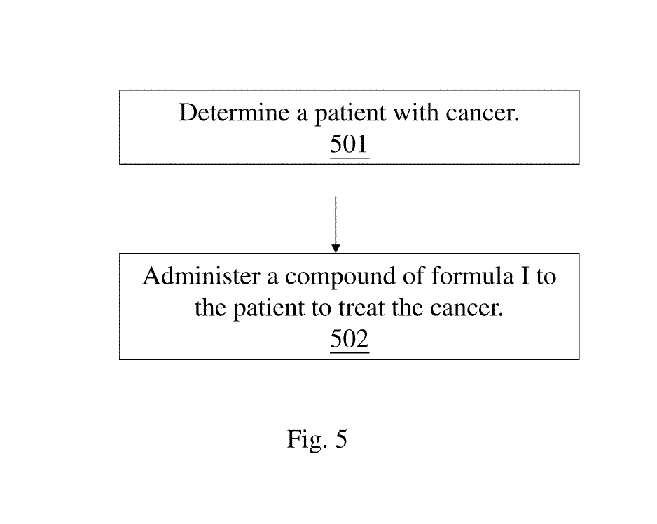

FIG. 5 is a method to treat cancer in a patient.

Block 501 states determine a patient with cancer.

In one example embodiment, the cancer is colon cancer. In another example embodiment, the patient does not has p53 mutation. In a further embodiment for example, the p53 mutation is deletion of p53. In another example embodiment, the colon cancer with deletion of p53 can be determined using commercially available methods.

Block 502 states administer the compound of Formula I to the patient to treat the cancer.

In one example embodiment, the compound is administered directly or in pharmaceutical compositions along with suitable carriers or excipients. In one example embodiment, suitable routes of administration may, for example, include oral, rectal, transmucosal, nasal, or intestinal administration and parenteral delivery. The compound or the pharmaceutical composition that includes the compound can be administered locally. For example, the compound can be delivered via injection or in a targeted drug delivery system, such as a depot or sustained release Formulation.

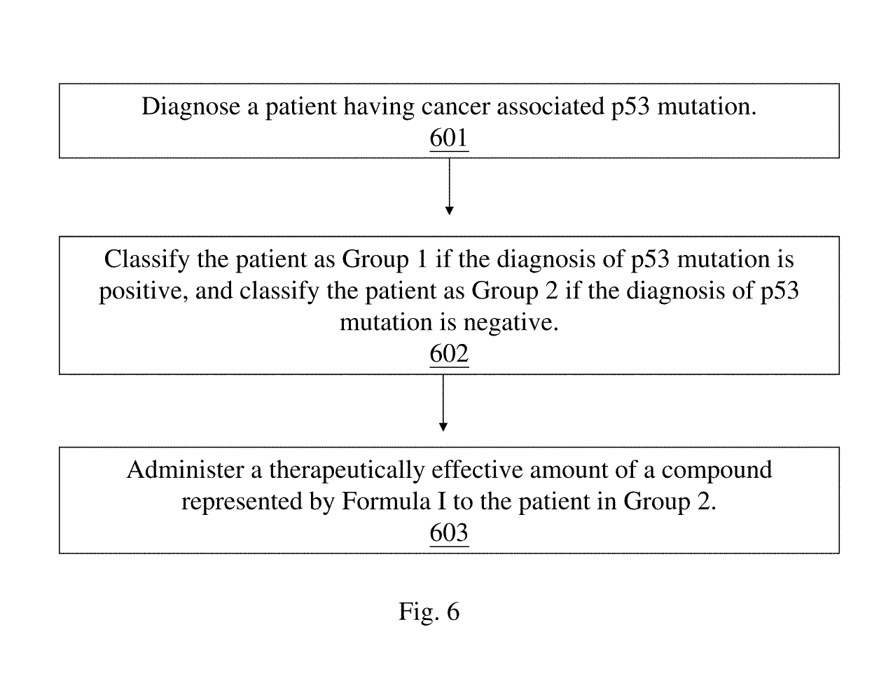

FIG. 6 is a method to treat cancer in a patient in need of such treatment in accordance with an example embodiment.

Block 601 states diagnose the patient having cancer associated p53 mutation. In an example embodiment, the p53 mutation is deletion of p53.

Block 602 states classify the patient as Group 1 if the diagnosis of p53 mutation is positive, and classify the patient as Group 2 if the diagnosis of p53 mutation is negative.

Block 603 states administer a therapeutically effective amount of the compound represented by Formula I to the patient in Group 2.

In one example embodiment, the compound is administered directly or in pharmaceutical compositions along with suitable carriers or excipients. In one example embodiment, suitable routes of administration may, for example, include oral, rectal, transmucosal, nasal, or intestinal administration and parenteral delivery. The compound or the pharmaceutical composition that includes the compound can be administered locally. For example, the compound can be delivered via injection or in a targeted drug delivery system, such as a depot or sustained release Formulation.

As used herein, the term “patient” encompasses mammals and non-mammals. Examples of mammals include, but are not limited to, humans, chimpanzees, apes monkeys, cattle, horses, sheep, goats, swine; rabbits, dogs, cats, rats, mice, guinea pigs, and the like. Examples of non-mammals include, but are not limited to, birds, fish and the like.

As used herein, the term “therapeutically effective amount” refers to any amount of a compound which, as compared to a corresponding patient who has not received such amount, results in improved treatment, healing, prevention, or amelioration of a disease, disorder, or side effect, or a decrease in the rate of advancement of a disease or disorder.

As used herein, the term “treat,” “treating” or “treatment” refers to methods of alleviating, abating or ameliorating a disease or condition symptoms, preventing additional symptoms, ameliorating or preventing the underlying metabolic causes of symptoms, inhibiting the disease or condition, arresting the development of the disease or condition, relieving the disease or condition, causing regression of the disease or condition, relieving a condition caused by the disease or condition, or stopping the symptoms of the disease or condition either prophylactically and/or therapeutically.

As used herein, the term “administration” or “administering” of the patient compound refers to providing a compound of an example embodiment and/or prodrugs thereof to a patient in need of treatment.

As used herein and in the claims, “comprising” means including the following elements but not excluding others.

1. A method of treating colon cancer in a patient in need thereof, comprising: administering a therapeutically effective amount of a compound to the patient to treat the colon cancer, wherein the compound is represented by Formula I 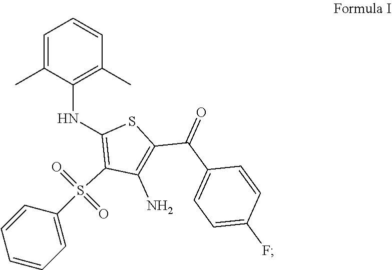 [SMILES: Cc1cccc(C)c1Nc1sc(C(=O)c2ccc(F)cc2)c(N)c1S(=O)(=O)c1ccccc1]
wherein said method comprises inducing apoptosis in the colon cancer without arresting cell cycle; and wherein the colon cancer does not have a p53 mutation.

2-4. (canceled)

5. A method of treating colon cancer in a patient in need thereof comprising:
diagnosing if the patient's colon cancer is associated with a p53 mutation;
classifying the patient as Group 1 if the colon cancer has a p53 mutation, and classifying the patient as Group 2 if the colon cancer does not have a p53 mutation (wild type);
administering a therapeutically effective amount of a compound to the patient in Group 2 to treat the colon cancer,
wherein said method comprises inducing apoptosis in the colon cancer without arresting cell cycle;
and wherein the compound is represented by Formula I:

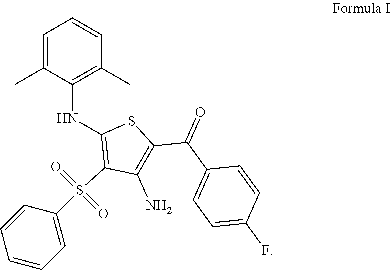 [SMILES: Cc1cccc(C)c1Nc1sc(C(=O)c2ccc(F)cc2)c(N)c1S(=O)(=O)c1ccccc1]

6-9. (canceled)
